(19) United States
(12) Patent Application Publication
(10) Pub. No.: US 2026/0177369 A1
(43) Pub. Date: Jun. 25, 2026

(54) APPARATUS FOR SENSING GAS AND DEFORMATION

(71) Applicant: INDUSTRIAL TECHNOLOGY RESEARCH INSTITUTE, Chutung (TW)

(72) Inventors: Hua-Lun CHI, Taichung City (TW); Yu-Jie SHEN, Taitung City (TW); Chien-Nan YEH, Tainan City (TW); Chao-Ta HUANG, Hsinchu City (TW)

(73) Assignee: INDUSTRIAL TECHNOLOGY RESEARCH INSTITUTE, Chutung (TW)

(21) Appl. No.: 19/190,133

(22) Filed: Apr. 25, 2025

(30) Foreign Application Priority Data
Dec. 23, 2024 (TW) 113148700

(51) Int. Cl. G01B 7/16; G01N 33/00; H01M 10/42

(52) U.S. Cl. CPC G01B 7/18; G01N 33/0027; H01M 10/4285

(57) ABSTRACT

An apparatus for detecting gas and deformation is provided, which includes a substrate, a case, an elastic portion, a deformation sensing element, a gas sensing element, and a protruding portion. The case includes an annular fixed portion having a lower surface, an annular connecting portion connecting the substrate and the annular fixed portion along an axis direction of a central axis passing through a center of the substrate, and an opening disposed on the annular connecting portion. The elastic portion connects to the annular fixed portion and is surrounded by the annular fixed portion. A cavity is defined by the substrate, the elastic portion and the annular connecting portion, and in communication with the opening. The deformation sensing element is disposed on the first surface. The protruding portion connects to the elastic portion, extends from the second surface along the central axis, and extends beyond the lower surface.

### CROSS REFERENCE TO RELATED APPLICATIONS

The present application is based on, and claims priority from, Taiwan (International) application No. 113148700, filed Dec. 13, 2024, the disclosure of which is hereby incorporated by reference herein in its entirety.

### TECHNICAL FIELD

The disclosure relates to a sensing apparatus, and, in particular, to an apparatus configured to detect gas and deformation.

### BACKGROUND

Current large-scale energy storage systems (such as at M W level) mainly include combination of multiple lithium batteries. Such energy storage systems typically include a lot of lithium battery cells (such as about thousands of cells to hundreds of thousands of cells). However, the battery management systems used for managing lithium batteries are generally designed to initiate remedial actions (such as disconnecting power) only after abnormal conditions (e.g., overvoltage, overcurrent, overheating) occur. Such system does not include pre-warning mechanisms.

Therefore, a sensing apparatus capable of real-time detection of battery health is required to effectively monitor the system's real-time operating status and perform predictive diagnostics to enhance the reliability and operational efficiency of the energy storage system.

### SUMMARY

An embodiment of the disclosure provides an apparatus configured to detect gas and deformation is provided, which includes a substrate, a case, an elastic portion, a deformation sensing element, at least one gas sensing element, and a protruding portion. The case includes an annular fixed portion, an annular connecting portion, and at least one opening. The annular fixed portion has a lower surface. The annular connecting portion connects the substrate and the annular fixed portion along an axis direction of a central axis, and the central axis passes through a center of the substrate. The opening is disposed on the annular connecting portion. The elastic portion connects to the annular fixed portion and is surrounded by the annular fixed portion. A cavity is defined by the substrate, the elastic portion and the annular connecting portion. The cavity is in communication with the opening. The deformation sensing element is disposed on the first surface. The protruding portion connects to the elastic portion, extends from the second surface of the elastic portion along the central axis, and extends beyond the lower surface of the annular fixed portion.

In some embodiments, the protruding portion includes a protruding surface, and a distance between a second surface of the elastic portion and the protruding surface in the axis direction is greater than a distance between the second surface of the elastic portion and the lower surface in the axis direction.

In some embodiments, the annular fixed portion surrounds a portion of the protruding portion to form a recess between the annular fixed portion and the protruding portion.

In some embodiments, the elastic portion includes a plurality of through holes penetrating the first surface and the second surface of the elastic portion to allow the cavity being in communication with the recess.

In some embodiments, a stiffness of the elastic portion along the axis direction is less than a stiffness of the annular fixed portion along the axis direction.

In some embodiments, a dimension of the elastic portion along the axis direction is less than a dimension of the annular fixed portion along the axis direction.

In some embodiments, the elastic portion includes a plurality of connecting ends, the elastic portion connects to the annular fixed portion through the plurality of connecting ends, and a plurality of gaps are formed between the elastic portion and the annular fixed portion.

In some embodiments, a stiffness of the elastic portion along the axis direction is less than a stiffness of the annular connecting portion along the axis direction.

In some embodiments, a stiffness of the elastic portion along the axis direction is less than a stiffness of the protruding portion along the axis direction.

In some embodiments, a stiffness of the elastic portion along the axis direction is less than a stiffness of the substrate along the axis direction.

In some embodiments, a distance between the opening and the lower surface of the substrate along the axis direction is less than a distance between the opening and the first surface of the elastic portion along the axis direction.

In some embodiments, the opening extends along an axis, the axis passes through the gas sensing element, and extends in a direction different from the direction of the central axis.

In some embodiments, the case further includes an outer surface, the lower surface and the outer surface face different directions, and the opening extends from the lower surface to the outer surface.

In some embodiments, the opening is L-shaped and includes a first portion and a second portion, the first portion extends in a direction perpendicular to the axis direction of the central axis, and the second portion extends in a direction parallel to the axis direction of the central axis.

In some embodiments, the apparatus further includes a columnar element disposed on the case and extending along the axis direction of the central axis, wherein the second portion of the opening is partially formed in the columnar element.

In some embodiments, the protruding portion, the elastic portion, and the deformation sensing element are sequentially arranged along the axis direction of the central axis.

In some embodiments, the substrate includes a substrate surface facing the deformation sensing element, and the gas sensing element is disposed on the substrate surface.

In some embodiments, the apparatus further includes a fastening element, the case further includes an inner surface and an outer surface, the inner surface faces the gas sensing element, and the fastening element is disposed on the outer surface.

In some embodiments, the fastening element, the substrate, and the protruding portion are sequentially arranged along the axis direction of the central axis.

In some embodiments, the case further includes a through hole extending along the central axis.

### BRIEF DESCRIPTION OF THE DRAWINGS

Aspects of the present disclosure are best understood from the following detailed description when read with the accompanying figures. It should be noted that, in accordance with the standard practice in the industry, various features are not drawn to scale. In fact, the dimensions of the various features may be arbitrarily increased or reduced for clarity of discussion.

FIG. 1 is a schematic view of a sensing apparatus in some embodiments of the disclosure.

FIG. 2 is a side view of the sensing apparatus in some embodiments of the disclosure.

FIG. 3 is a cross-sectional view of the sensing apparatus in some embodiments of the disclosure.

FIG. 4 is a partial cross-sectional view of the sensing apparatus in some embodiments of the disclosure.

FIG. 5 is a perspective view of the sensing apparatus in other embodiments of the disclosure.

FIG. 6 is a schematic view of some elements of the sensing apparatus.

FIG. 7 is a schematic view of an energy storage system in some embodiments of the disclosure.

FIG. 8 is a partial schematic view of the energy storage system in some embodiments of the disclosure.

### DETAILED DESCRIPTION

The following disclosure provides many different embodiments, or examples, for implementing different features of the provided subject matter. Specific examples of components and arrangements are described below to simplify the disclosure. These are, of course, merely examples and are not intended to be limiting.

Spatially relative terms, such as “up,” “down,” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

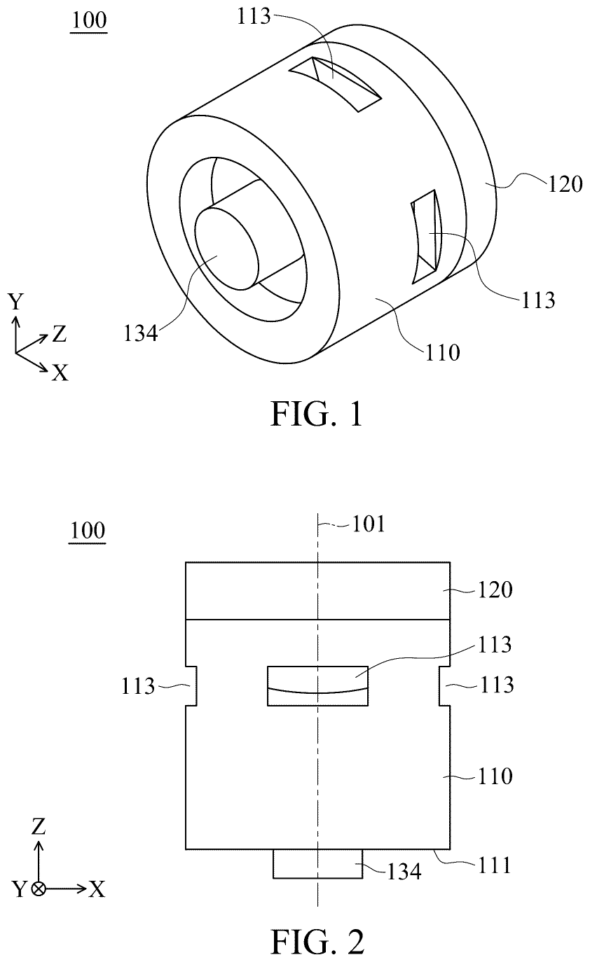

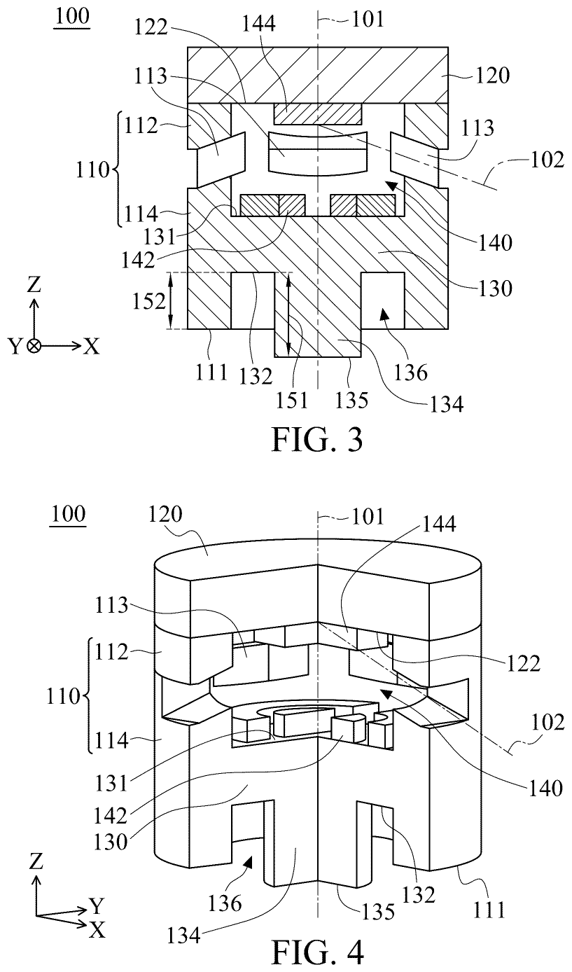

A sensing apparatus is provided in some embodiments of the present disclosure, in particular, an apparatus configured to detect gas and deformation to enable real-time monitoring of a battery module's health. For example, FIG. 1 is a schematic view of a sensing apparatus 100 in some embodiments of the disclosure. FIG. 2 is a side view of the sensing apparatus 100 in some embodiments of the disclosure. FIG. 3 is a cross-sectional view of the sensing apparatus 100 in some embodiments of the disclosure. FIG. 4 is a partial cross-sectional view of the sensing apparatus 100 in some embodiments of the disclosure. As shown in FIG. 1 to FIG. 4, the sensing apparatus 100 includes a case 110, a substrate 120, and an elastic portion 130 arranged along a central axis 101. The case 110 connects the substrate 120 and the elastic portion 130. The central axis 101 extends in a Z direction and passes through a center (such as the geometric center) of the substrate 120. The substrate 120, an annular connecting portion 112, and the elastic portion 130 define a cavity 140. A deformation sensing element 142 and a gas sensing element 144 are disposed in the cavity 140 and configured to detect deformation (such as in a lithium battery) and gas, respectively. For example, the deformation sensing element 142 may include a strain gauge, and the gas sensing element 144 may include a gas sensing material, a heater, and sensing electrodes. Since the deformation sensing element 142 and the gas sensing element 144 are disposed in the same cavity 140, the overall volume of the sensing apparatus 100 may be reduced to achieve miniaturization.

The elastic portion 130 may have a first surface 131 and a second surface 132 facing different directions (such as may face opposite directions). For example, the first surface 131 may face the positive direction of the Z axis and face the substrate 120, the deformation sensing element 142, and the gas sensing element 144. The second surface 132 may face the negative direction of the Z axis. The elastic portion 130 may include a protruding portion 134 that extends from the second surface 132 along the central axis 101, such as extending in the negative direction of the Z axis. The deformation sensing element 142 may be disposed on the first surface 131 of the elastic portion 130. In other words, the protruding portion 134 and the deformation sensing element 142 may be disposed on opposite sides of the elastic portion 130. In some embodiments, in the direction along the central axis 101, the protruding portion 134, the elastic portion 130, and the deformation sensing element 142 may be arranged along the central axis 101, so that an external force applied to the sensing apparatus 100 may be effectively transmitted from the protruding portion 134 to the elastic portion 130 in the direction extending along the central axis 101, and the force may be detected by the deformation sensing element 142. However, the disclosure is not limited thereto. The deformation sensing element 142 may be disposed on other positions, depending on application requirements.

In some embodiments, the protruding portion 134 may protrude beyond the case 110. For example, the case 110 may have a lower surface 111 facing in the negative direction of the Z axis, and the protruding portion 134 may protrude beyond the lower surface 111. In other words, as shown in FIG. 2, the protruding portion 134 may be at least partially exposed from the case 110. Alternatively, as shown in FIG. 3, the protruding portion 134 may have a protruding surface 135 facing in the negative direction of the Z axis. The central axis 101 may extend along the Z axis, and a distance 151 between the second surface 132 and the protruding surface 135 may be greater than a distance 152 between the second surface 132 and the lower surface 111 in the direction along the central axis 101. Therefore, the protruding portion 134 may be pressed against a device (such as the energy storage unit) whose deformation is to be detected. If the device deforms and expends, a force in the Z direction will be applied to the protruding portion 134, and then the force may be applied on the elastic portion 130 through the protruding portion 134 to deform the elastic portion 130. The deformation sensing element 142 disposed on the elastic portion 130 may be used to detect the amount of deformation of the elastic portion 130 to determine the extent of deformation of the aforementioned device. As a result, the user may integrate this deformation with the sensing result of the gas sensing element 144 to determine the health status of the energy storage unit in real time.

In some embodiments, the case 110 may include an annular connecting portion 112 and an annular fixed portion 114. The annular connecting portion 112 may connect the substrate 120 and the annular fixed portion 114 along the axis direction (such as the Z direction) of the central axis 101. The annular fixed portion 114 may connect to the elastic portion 130, and may surround a portion of the elastic portion 130 and the protruding portion 134 to form a recess 136 between the annular fixed portion 114 and the protruding portion 134. The lower surface 111 may be formed on the annular fixed portion 114. In order to minimize the deformation of the case 110 when the elastic portion 130 is pushed by an external device through the protruding portion 134, the stiffness of the elastic portion 130 along the axis direction (e.g., the Z direction) of the central axis 101 may be less than the stiffness of the annular fixed portion 114 or the annular connecting portion 112 along the axis direction. In other words, the deformations of the annular fixed portion 114 and the deformations of the annular connecting portion 112 are respectively smaller than the deformation of the elastic portion 130 under a same axial force in the Z direction. In this way, the sensing results of the deformation sensing element 142 may not be interfered by the deformation of the case 110, enhancing the sensing accuracy of the sensing apparatus 100 for external force detection. In some embodiments, the dimension (such as thickness) of the elastic portion 130 along the axis direction of the central axis may also be smaller than the dimension of the annular fixed portion 114 or be smaller than the thickness of the annular connecting portion 112 along the axis direction. In some embodiments, the case 110 and the elastic portion 130 may be designed to include different materials, and these materials have different stiffnesses along the axis direction of the central axis. For example, the material of the case 110 may have a greater stiffness along the axis direction than the material of the elastic portion 130 to achieve a similar effect.

In some embodiments, other elements may also be designed according to design principles similar to aforementioned design principles to further eliminate the influence of other elements on deformation sensing. For example, in some embodiments, the stiffness of the elastic portion 130 along the axis direction of the central axis 101 may be designed to be less than the stiffness of the protruding portion 134 and the stiffness of the substrate 120 along the axis direction of the central axis 101. Therefore, when external forces are transmitted to the elastic portion 130, excessive deformation of other elements which significantly affects the sensing accuracy and sensitivity of the deformation sensing element 142 may be avoided.

In some embodiments, the gas sensing element 144 may be disposed on the substrate 120. The substrate 120 may have a substrate surface 122 facing the deformation sensing element 142, and the gas sensing element 144 is disposed on the substrate surface 122. In addition, at least one opening 113 may be provided on the annular connecting portion 112 of the case 110. The opening 113 is in communication with the cavity 140 to allow external gas such as air to pass through the opening 113 and reach the gas sensing element 144 disposed in the cavity 140. In some embodiments, the opening 113 may be designed to extend along an axis 102, wherein the axis 102 may pass through the gas sensing element 144 and extend in a direction different from the central axis 101. Alternatively, the opening 113 may be provided at any position on the case 110 between the annular connecting portion 112 and the annular fixed portion 114. As a result, when external air flows through the opening 113 into the cavity 140, the air may reach the gas sensing element 144 more quickly to achieve better sensing performance. Furthermore, when the sensing apparatus 100 is provided with the substrate 120 facing upward and the protruding portion 134 facing downward, lighter gas (such as hydrogen) entering the cavity 140 through the opening 113 may flow upwardly to ensure it is easier for the gas sensing element 144 to detect the gas. This increases the sensitivity and accuracy of the sensing apparatus 100 when detecting gas.

Moreover, by designing the opening 113 on the annular connecting portion 112, the heat generated by the heater in the gas sensing element 144 may be dissipated from the cavity 140 by thermal convection effect. Therefore, the heat will not induce thermal stress on the deformation sensing element 142 to avoid affecting the measurement accuracy of the deformation sensing element 142. In addition, the opening 113 also prevents the heat generated by the heat dissipater within the gas sensing element 144 from being transferred to the deformation sensing element 142 via thermal conduction effect. Consequently, this heat will not generate thermal stress on the deformation sensing element 142 to prevent the measurement accuracy of the deformation sensing element from being affected.

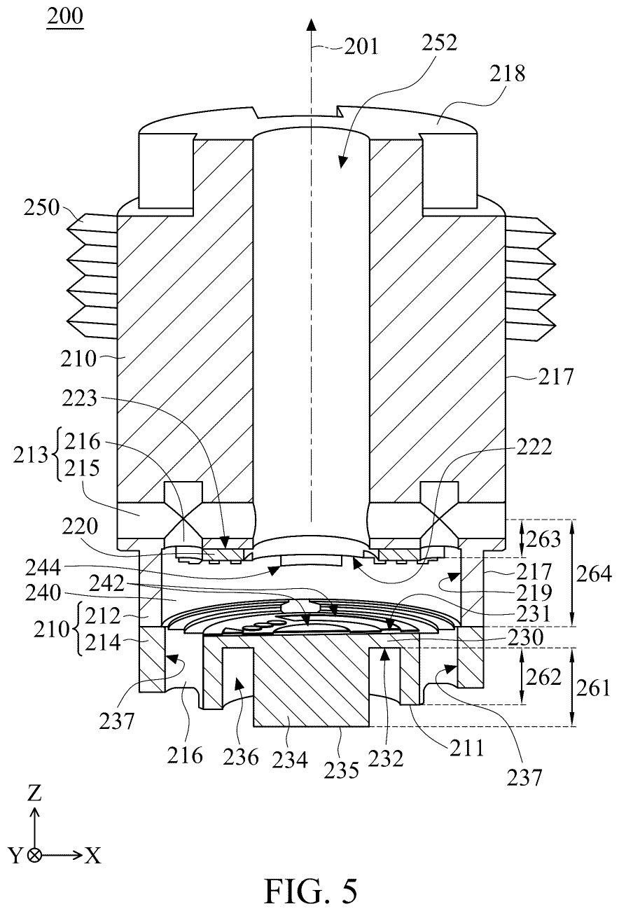

Although aforementioned embodiments provide some configurations of the sensing apparatus, the disclosure is not limited thereto. For example, FIG. 5 is a perspective view of a sensing apparatus 200 according to other embodiments of the disclosure. As shown in FIG. 5, the sensing apparatus 200 includes a case 210, a substrate 220, and an elastic portion 230 arranged along a central axis 201. The case 210 may be connected to the substrate 220 and the elastic portion 230. The central axis 201 extends in the Z direction and passes through a center (such as the geometric center) of the case 210. In addition, a cavity 240 is defined by an annular connecting portion 212 of the case 210, the elastic portion 230, and the substrate 220. In other words, the cavity 240 is surrounded by the annular connecting portion 212, the elastic portion 230, and the substrate 220. A deformation sensing element 242 and a gas sensing element 244 may be disposed in the cavity 240 and configured to detect deformation and gas, respectively. For example, the deformation sensing element 242 may include a strain gauge, and the gas sensing element 244 may include a gas sensing material, a heater, and sensing electrodes. Since the deformation sensing element 242 and the gas sensing element 244 are disposed in the same cavity 240, the overall volume of the sensing apparatus 200 may be reduced to achieve miniaturization.

The elastic portion 230 may have a first surface 231 and a second surface 232 facing in different directions. For example, the first surface 231 may face the positive direction of the Z axis, such as facing the substrate 220, the deformation sensing element 242, and the gas sensing element 244, while the second surface 232 may face the negative direction of the Z axis. A protruding portion 234 extending from the second surface 232 along the central axis 201 may be disposed on the elastic portion 230, such as extending in the negative direction of the Z axis, and the deformation sensing element 242 may be disposed on the first surface 231 of the elastic portion 230. In other words, the protruding portion 234 and the deformation sensing element 242 may be disposed on two opposite surfaces of the elastic portion 230. In some embodiments, the protruding portion 234, the elastic portion 230, and the deformation sensing element 242 may be sequentially arranged along the central axis 201 to effectively transmit an external force applied to the sensing apparatus 200 from the protruding portion 234 to the elastic portion 230, and then the force is detected by the deformation sensing element 242. However, the disclosure is not limited thereto, and the deformation sensing element 242 may be disposed in other positions, depending on the force applied on the object to be detected.

In some embodiments, the protruding portion 234 may protrude beyond the case 210. For example, the case 210 may have a lower surface 211 facing the negative direction of the Z axis, and the protruding portion 234 may protrude beyond the lower surface 211. In other words, as shown in FIG. 5, when viewed from the direction of the Y axis, the protruding portion 234 may be at least partially exposed from the case 210. The protruding portion 234 may have a protruding face 235 that faces the negative direction of the Z axis. The central axis 201 may extend along the Z axis, and a distance 261 between the second surface 232 and the protruding face 235 may be greater than a distance 262 between the second surface 232 and the lower surface 211 in the axis direction that the central axis 201 extends. As a result, the protruding portion 234 may be pressed against the device whose deformation is to be detected (such as an energy storage unit). If the device undergoes deformation and expansion, a force will be generated in the positive direction of the Z axis and applied to the protruding portion 234. The force applied to the protruding portion 234 is transmitted through the protruding portion 234 to the elastic portion 230 to deform the elastic portion 230. The deformation sensing element 242 disposed on the elastic portion 230 is used to detect the amount of deformation of the elastic portion 230, and then determining the extent of deformation of the device. As a result, the user may integrate this deformation with the sensing result of the gas sensing element 244 to determine the health status of the energy storage unit in real time.

In some embodiments, the case 210 may include an annular connecting portion 212 and an annular fixed portion 214, and the annular connecting portion 212 may connect the substrate 220 and the annular fixed portion 214 along the axis direction (e.g., the Z direction) of the central axis 201. The annular fixed portion 214 connects to the elastic portion 230 and may surround a portion of the elastic portion 230 and the protruding portion 234 to form a recess 236 between the annular fixed portion 214 and the protruding portion 234. The lower surface 211 may be formed on the annular fixed portion 214. When an external device pushes the elastic portion 230 through the protruding portion 234, the stiffness of the elastic portion 230 along the axis direction (such as the Z direction) of the central axis 201 may be lower than the stiffness of the annular fixed portion 214 and the stiffness of the annular connecting portion 212 along the axis direction to minimize deformation of the case 210. In other words, the annular fixed portion 214 and the annular connecting portion 212 of the case 210 are less deformable when compared to the elastic portion 230, so as to prevent the deformation of the case 210 from interfering with the measurement accuracy of the deformation sensing element 242. Furthermore, in some embodiments, the dimension (such as the thickness) of the elastic portion 230 along the axis direction of the central axis 201 may be less than the dimension of the annular fixed portion 214 and the dimension of the annular connecting portion 212 along the axis direction. In some embodiments, the case 210 and the elastic portion 230 may include different materials, and these materials have different stiffnesses. For example, the material of the case 210 may have greater stiffness than the material of the elastic portion 230 along the axis direction of the central axis to achieve a similar effect.

In some embodiments, to further eliminate the influence of other elements on deformation sensing, other elements may also be designed according to similar principles as described above. For example, in some embodiments, the stiffness of the elastic portion 230 along the axis direction of the central axis 201 may be designed to be lower than the stiffness of the protruding portion 234 along the axis direction of the central axis 201. As a result, when external forces are applied to the elastic portion 230, excessive deformation of other elements, which significantly affects the measurement accuracy and sensitivity of the deformation sensing element 242, may be prevented.

In some embodiments, a plurality of through holes (not shown) may be disposed on the elastic portion 230 to allow the cavity 240 to in communicate with the external environment. Specifically, the through holes may extend along the central axis 201 and penetrate the first surface 231 and the second surface 232 of the elastic portion 230. Therefore, the number of channels connecting the cavity 240 to the outside may be increased, so the heat dissipation capability of the cavity 240 may be enhanced by thermal convection effect. The through holes (not shown) allow the heat generated by the heater within the gas sensing element 244 to be dissipated from the cavity 240 via thermal convection effect. As a result, the heat does not induce thermal stress on the deformation sensing element 242 to avoid affecting the measurement accuracy of the deformation sensing element 242. In addition, the through holes 233 (not shown) may also prevent the heat dissipated by the heat sink in the gas sensing element 244 from being transferred to the deformation sensing element 242 by thermal conduction effect, so the thermal isolation of the deformation sensing element 242 may be enhanced. Therefore, the heat generated by the gas sensing element will not induce thermal stress on the deformation sensing element 242, so the measurement accuracy of the deformation sensing element 242 may be prevented from being affected. Furthermore, providing through holes (not shown) on the elastic portion 230 may reduce the stiffness of the elastic portion 230 along the axis direction (the direction that the Z axis extends), thereby increasing the measurement sensitivity of the deformation sensing element 242.

In some embodiments, as shown in FIG. 5, the elastic portion 230 may include a plurality of connecting ends 237, and the elastic portion 230 is connected to the annular fixed portion 214 through the connecting ends 237. As a result, a plurality of slots (not shown) may be formed between the elastic portion 230 and the annular fixed portion 214. This configuration may reduce the stiffness of the elastic portion 230 along the axis direction of the central axis 201 (the Z direction), so the measurement sensitivity of the deformation sensing element 242 may be increased. Moreover, providing a plurality of connecting ends 237 on the elastic portion 230 and forming a plurality of slots between the elastic portion 230 and the annular fixed portion 214 may also enhance the thermal isolation of the elastic portion 230. Therefore, this prevents a large amount of heat from being conducted from the gas sensing element 244 to the deformation sensing element 242, and then avoids the thermal stress generated on the deformation sensing element 242. As a result, the measurement accuracy of the deformation sensing element may be improved.

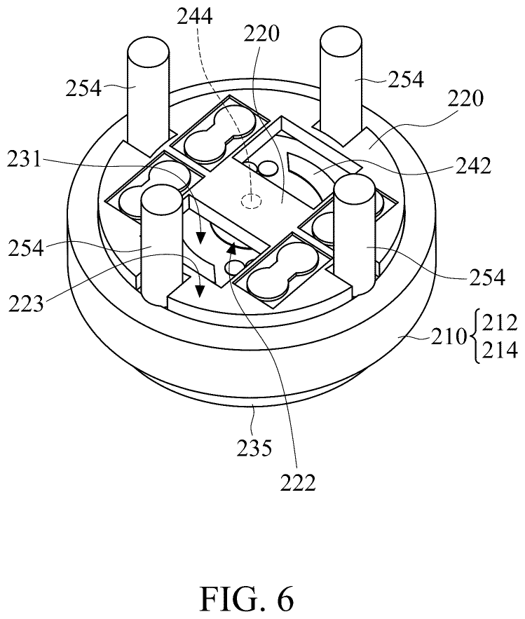

FIG. 6 is a schematic view of some elements of the sensing apparatus 200 after assembly, wherein a portion of the case 210 is omitted to more clearly illustrate the positional relationships of the other elements. In some embodiments, as shown in FIG. 5 and FIG. 6, the gas sensing element 244 may be disposed on the substrate 220. The substrate 220 may have a first substrate surface 222 facing the deformation sensing element 242 and a second substrate surface 223 facing in the opposite direction to the first substrate surface 222. As shown in FIG. 6, the gas sensing element 244 may be disposed on the first substrate surface 222, the deformation sensing element 242 is disposed on the first surface 231 of the elastic portion 230, and the first surface 231 faces the gas sensing element 244 and the first substrate surface 222.

Furthermore, in some embodiments, as shown in FIG. 5 and FIG. 6, a plurality of columnar elements 254 and openings 213 may be disposed on the case 210. The columnar elements 254 may extend along the Z axis. The openings 213 may be disposed on the annular connecting portion 212 or on a portion of the case 210 above the annular connecting portion 212 (as shown in FIG. 5), and the openings 213 may be in communication with the cavity 240 to allow external air to pass through the opening 213 and reach the gas sensing element 244 disposed within the cavity 240 to enhance the sensing sensitivity of the gas sensing element 244. By providing the columnar elements 254 extending along the Z axis, the stiffness of the annular connecting portion 212 along the axial (Z axis) direction may be increased to prevent the measurement sensitivity of the deformation sensing element 242 being reduced. In addition, the columnar elements 254 extending along the Z axis may also prevent the torsional rigidity of the case 210 about the central axis 201 being reduced. As a result, excessive twisting of the case 210 due to torque may be prevented, and unnecessary torsional deformation of the elastic portion 230 may thus be prevented. Therefore, the measurement accuracy of the deformation sensing element 242 may be maintained.

In some embodiments, the opening 213 may be L-shaped, such as may include a first portion 215 that extends in the XY plane (in a direction perpendicular to the central axis 201) and a second portion 216 that extends along the Z axis. Moreover, the second portion 216 of the opening 213 may be partially formed in the columnar element 254. When the second portion 216 of the opening 213 may be partially formed within the columnar element 254, it may not only prevent a significant reduction in the stiffness of the annular fixed portion 214 along the axis direction of the central axis 201, but also prevent a significant reduction in the resistance to twisting of the case 210. Furthermore, when the second portion 216 of the opening 213 may be partially formed in the columnar element 254, not only the sensing sensitivity of the gas sensing element 244 may be enhanced, but also the measurement accuracy of the deformation sensing element 242 may be ensured. The case 210 may have an outer surface 217 and an inner surface 219 facing in directions different from the direction that the lower surface 211 faces, and the inner surface 219 faces the gas sensing element 244. The first portion 215 of the opening 213 may extend from the outer surface 217 into the case 210, and then connect to the second portion 216 of the opening 213. Afterwards, the second portion 216 may extend from the cavity 240 to the lower surface 211 of the case 210. As a result, external air may reach the gas sensing element 244 disposed in the cavity 240 through the opening 213.

In addition, by providing the opening 213 on the annular connecting portion 212 or on the portion of the case 210 above the annular connecting portion 212, the heat generated by the heater in the gas sensing element 244 may be dissipated from the cavity 240 via thermal convection effect. Therefore, the heat will not induce the thermal stress on the deformation sensing element 242 to prevent affecting the measurement accuracy of the deformation sensing element 242. Moreover, the opening 213 also prevents the heat generated by the heat spreader in the gas sensing element 244 from being transferred to the deformation sensing element 242 via thermal conduction effect. so the measurement accuracy of the deformation sensing element 242 is prevented from being affected.

In some embodiments, as shown in FIG. 5, a distance 263 between the opening 213 and the lower surface 221 of the substrate 220 is less than a distance 264 between the opening 213 and the first surface 231 of the elastic portion 230 along the axis direction that the central axis 201 extends (the Z direction). Therefore, the opening 213 is positioned closer to the gas sensing element 244 disposed on the substrate 220 to enhance the thermal convection effect, so that the heat generated by the gas sensing element 244 may be more easily removed from the cavity 240. Additionally, disposing the opening 213 in this manner also enhances the thermal convection effect between the cavity 240 and the external environment, which prevents the gas detected by the gas sensing element 244 from continuously accumulating in the cavity 240 to avoid the gas concentration in the cavity 240 from becoming too high. As a result, the measurement accuracy of the gas sensing element 244 is enhanced.

In some embodiments, a fastening element 250 may be disposed on the outer surface 217 to affix the sensing apparatus 200 to other external equipment. For example, the fastening element 250 may include screw threads, adhesive, clips, etc., or it may be integrally formed with the case 210, or the fastening element 250 and the case 210 may be two separate elements. The fastening element 250, the substrate 220, and the protruding portion 234 are sequentially arranged along the axis direction of the central axis 201.

In some embodiments, the case 210 may also include a through hole 252 that extends along the central axis 201. The through hole 252 may extend from the upper surface 218 of the case 210 into the cavity 240, wherein the upper surface 218 and the lower surface 211 of the case 210 face opposite directions. Therefore, other elements may extend into the cavity 240 through the through hole 252, such as a conductive element (not shown) which is electrically connected to the deformation sensing element 242 and the gas sensing element 244 from the exterior of the sensing apparatus 200.

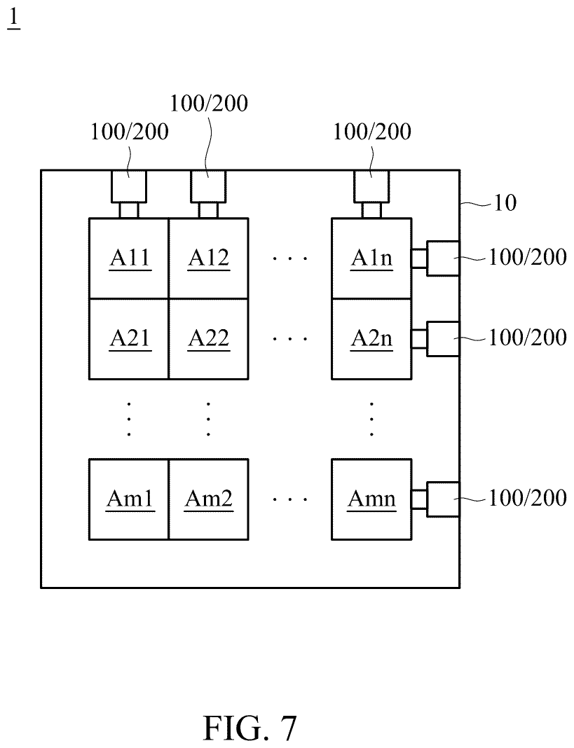

Various examples of applying the aforementioned sensing apparatus 100 or sensing apparatus 200 are provided below. FIG. 7 is a schematic view of an energy storage system 1 in some embodiments of the disclosure. As shown in FIG. 7, the energy storage system 1 may include a housing 10 and energy storage units Amn disposed in the housing 10 and arranged in a matrix, wherein m represents the column number and n represents the row number that the energy storage units located. Both m and n are positive integers, which may be equal or different. Some rows and columns are omitted in FIG. 7. For example, energy storage unit A11 represents the energy storage unit located in the first column and first row, energy storage unit A21 represents the energy storage unit located in the second column and first row, energy storage unit Am1 represents the energy storage unit located in the m-th column and first row, and energy storage unit Am2 represents the energy storage unit located in the m-th column and second row, and so on. Sensing apparatus 100 or sensing apparatus 200 may be disposed in each row and each column. For example, the protruding portions 134 or 234 of the sensing apparatus 100 or the sensing apparatus 200 may contact the energy storage units at the edge, such as the energy storage units A11, A12, A1n, and the energy storage units A1n, A2n, Amn, and another end of the sensing apparatus 100 or the sensing apparatus 200 (such as the aforementioned upper surface 218) contacts the housing 10. Alternatively, the sensing apparatus 100 or the sensing apparatus 200 is fixed to the housing 10 by the aforementioned fastening element 250.

The energy storage units Amn may include batteries, such as lithium batteries. When any energy storage unit Amn encounters a problem, it typically releases gas or undergoes deformation (such as expansion). Therefore, if any energy storage unit Amn experiences an abnormal issue, the sensing apparatus 100 or the sensing apparatus 200 may detect the abnormal gas released from the energy storage unit Amn, thereby providing a rapid early warning. Alternatively, when one energy storage unit Amn expands, the stress detected by the sensing apparatuses in that specific row and specific column will be different. For example, if energy storage unit A22 expands, it will push the energy storage units in the second row and the second column to cause the deformation values detected by each sensing apparatus 100 or 200 in that row and column to change. This enables precise localization of the problem energy storage unit. As a result, maintenance worker may only replace the problem energy storage unit in specific position to maintenance repair costs. In summary, by the combined operation of the deformation sensing element and the gas sensing element in the aforementioned sensing apparatus, abnormalities may be detected quickly and the location of the abnormal energy storage unit may be accurately determined.

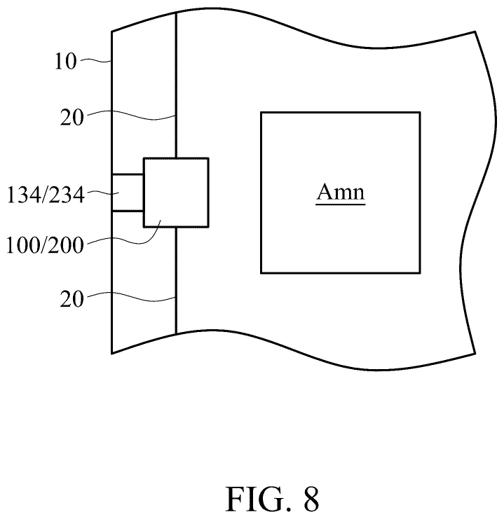

In addition, the sensing apparatus 100 or sensing apparatus 200 may also be used to detect deformation of the housing 10. For example, FIG. 8 is a partial schematic view of the energy storage system in some embodiments of the disclosure, wherein the sensing apparatus 100 or the sensing apparatus 200 may contact the housing 10 by the protruding portion 134 or the protruding portion 234. The energy storage system may also include a fixed portion 20, so that the sensing apparatus 100 or the sensing apparatus 200 may be disposed on the fixed portion 20. For example, the sensing apparatus 100 or sensing apparatus 200 may be affixed on the fixed portion 20 by the aforementioned fastening element 250. With this configuration, when the housing 10 deforms due to external forces, such deformation may be detected in real time to protect the energy storage units disposed in the housing 10 to enhance the safety of the energy storage system. Moreover, the sensing apparatus 100 or the sensing apparatus 200 may simultaneously detect any abnormal gas released by an energy storage unit Amn at this moment to detect the deformation of the housing 10 and detect abnormalities of the energy storage unit Amn at a same time.

In summary, an apparatus configured to detect gas and deformation is provided, which includes a case, a substrate, an elastic portion, a protruding portion, a deformation sensing element, and a gas sensing element. The case includes an opening and a lower surface, with a central axis passing through the center of the case. The substrate and the elastic portion are connected to the case. The elastic portion includes a first surface and a second surface, and the first surface and the second surface face different directions. The substrate, a portion of the case, and the elastic portion define a cavity, and the cavity is in communicate with the opening. The protruding portion extends from the first surface along the central axis and protrudes beyond the lower surface. The deformation sensing element is disposed on the second surface. The gas sensing element is disposed on the substrate and disposed in the cavity. Therefore, gas and deformation may be detected simultaneously to enable warning of the energy storage unit's health status through different sensing methods, which increases safety and reduces costs. Moreover, by integrating the deformation sensing element and the gas sensing element in the same case, the overall volume of the sensing device may be reduced to achieve miniaturization and lower process costs. The disclosure provides a real-time battery health monitoring sensing apparatus to effectively track the system's operating conditions and performing predictive diagnostics to enhance the reliability and operational efficiency of the energy storage system.

Although embodiments of the disclosure and their advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the invention of the disclosure, processes, machines, manufacture, compositions of matter, means, methods, or steps, presently existing or later to be developed, that perform substantially the same function or achieve substantially the same result as the corresponding embodiments described herein may be utilized according to the disclosure. Accordingly, the appended claims are intended to include within their scope of such processes, machines, manufacture, and compositions of matter, means, methods, or steps. In addition, each claim constitutes a separate embodiment, and the combination of various claims and embodiments are within the scope of the invention.

What is claimed is:

1. A apparatus configured for sensing gas and deformation, comprising:
a substrate;
a case, comprising:
an annular fixed portion having a lower surface;
an annular connecting portion connecting the substrate and the annular fixed portion along an axis direction of a central axis, wherein the central axis passes through a center of the substrate; and
at least one opening disposed on the annular connecting portion;
an elastic portion connecting to the annular fixed portion and surrounded by the annular fixed portion, wherein a cavity is defined by the substrate, the elastic portion and the annular connecting portion, and the cavity is in communication with the opening;
at least one deformation sensing element disposed on a first surface of the elastic portion;
at least one gas sensing element disposed on the substrate and in the cavity; and
a protruding portion connecting to the elastic portion, wherein the protruding portion extends from the first surface of the elastic portion along the central axis, and extends beyond the lower surface of the annular fixed portion.

2. The apparatus as claimed in claim 1, wherein the protruding portion comprises a protruding surface, and a distance between a second surface of the elastic portion and the protruding surface in the axis direction is greater than a distance between the second surface of the elastic portion and the lower surface in the axis direction.

3. The apparatus as claimed in claim 2, wherein the annular fixed portion surrounds a portion of the protruding portion to form a recess between the annular fixed portion and the protruding portion.

4. The apparatus as claimed in claim 3, wherein the elastic portion comprises a plurality of through holes penetrating the first surface and the second surface of the elastic portion to allow the cavity being in communication with the recess.

5. The apparatus as claimed in claim 1, wherein a stiffness of the elastic portion along the axis direction is less than a stiffness of the annular fixed portion along the axis direction.

6. The apparatus as claimed in claim 5, wherein a dimension of the elastic portion along the axis direction is less than a dimension of the annular fixed portion along the axis direction.

7. The apparatus as claimed in claim 5, wherein the elastic portion comprises a plurality of connecting ends, the elastic portion connects to the annular fixed portion through the plurality of connecting ends, and a plurality of gaps are formed between the elastic portion and the annular fixed portion.

8. The apparatus as claimed in claim 1, wherein a stiffness of the elastic portion along the axis direction is less than a stiffness of the annular connecting portion along the axis direction.

9. The apparatus as claimed in claim 1, wherein a stiffness of the elastic portion along the axis direction is less than a stiffness of the protruding portion along the axis direction.

10. The apparatus as claimed in claim 1, wherein a stiffness of the elastic portion along the axis direction is less than a stiffness of the substrate along the axis direction.

11. The apparatus as claimed in claim 1, wherein a distance between the opening and the lower surface of the substrate along the axis direction is less than a distance between the opening and the first surface of the elastic portion along the axis direction.

12. The apparatus as claimed in claim 1, wherein the opening extends along an axis, the axis passes through the gas sensing element and extends in a direction different from the direction of the central axis.

13. The apparatus as claimed in claim 1, wherein the case further comprises an outer surface, the lower surface and the outer surface face different directions, and the opening extends from the lower surface to the outer surface.

14. The apparatus as claimed in claim 13, wherein the opening is L-shaped and comprises a first portion and a second portion, the first portion extends in a direction perpendicular to the axis direction of the central axis, and the second portion extends in a direction parallel to the axis direction of the central axis.

15. The apparatus as claimed in claim 14, further comprising a columnar element disposed on the case and extending along the axis direction of the central axis, wherein the second portion of the opening is partially formed in the columnar element.

16. The apparatus as claimed in claim 1, wherein the protruding portion, the elastic portion, and the deformation sensing element are sequentially arranged along the axis direction of the central axis.

17. The apparatus as claimed in claim 1, wherein the substrate comprises a substrate surface facing the deformation sensing element, and the gas sensing element is disposed on the substrate surface.

18. The apparatus as claimed in claim 1, further comprising a fastening element, wherein the case further comprises an inner surface and an outer surface, the inner surface faces the gas sensing element, and the fastening element is disposed on the outer surface.

19. The apparatus as claimed in claim 18, wherein the fastening element, the substrate, and the protruding portion are sequentially arranged along the axis direction of the central axis.

20. The apparatus as claimed in claim 1, wherein the case further comprises a through hole extending along the central axis.
